(19) United States
(12) Patent Application Publication
(10) Pub. No.: US 2008/0206366 A1
(43) Pub. Date: Aug. 28, 2008

(54) COSMETIC COMPOSITION COMPRISING EXTRACT FROM MALLOTUS JAPONICUS FOR IMPROVING SKIN WRINKLE

(76) Inventors: Jung No LEE, Chungcheongnam-do (KR); Jee Hean JEONG, Suwon-shi (KR); Byong Kee JO, Anyang-shi (KR); Kang-Tae LEE, Cheonan-shi (KR)

Correspondence Address: NIXON & VANDERHYE, PC, 901 NORTH GLEBE ROAD, 11TH FLOOR, ARLINGTON, VA, 22203, US

(21) Appl. No.: 12/112,506

(22) Filed: Apr. 30, 2008

Related U.S. Application Data
(62) Division of application No. 10/575,869, filed on Apr. 14, 2006.
Application No. 10/575,869 is a continuation of application No. PCT/KR2004/000881, filed on Apr. 16, 2004.

(30) Foreign Application Priority Data
Oct. 24, 2003 (KR) 10-2003-0074764

(51) Int. Cl. A61K 36/00; A61Q 19/00

(52) U.S. Cl. 424/725

(57) ABSTRACT

The present invention relates to compositions for inhibiting elastase activity, enhancing collagen biosynthesis and improving skin wrinkle, comprising Mallotus japonicus extract as active ingredient. The present compositions exhibit the inhibition effect on elastase activity and promotion effect on collagen biosynthesis at a molecular level, and as a result, excellent efficacy in improvement of skin wrinkles.

The present application is a divisional of application Ser. No. 10/575,869, filed Apr. 14, 2006 which is a 371 U.S. National Phase of PCT/KR04/00881, filed Apr. 16, 2004, which claims benefit of KR 10-2003-0074764, filed Oct. 24, 2003, the entire contents of each of which is hereby incorporated herein by reference.

### BACKGROUND OF THE INVENTION

1. Field of the Invention

The present invention relates to compositions for inhibiting elastase activity, enhancing collagen biosynthesis and improving skin wrinkle.

2. Description of the Related Art

Human skin shows its declined functions with aging due to various internal and external factors. In accordance with internal aspects of skin aging, the activity and biosynthesis of the constituents forming body exhibit decreased pattern due to lowered activity of cell and active oxygen species attack the cell constituents to lead to their deteriorative roles in terms of structure and function. In accordance with external aspects of skin aging, external factors surrounding skin such as ultraviolet ray are responsible for skin drying, lowering of skin elasticity and deposition of pigments. In particular, strong irritation associated with ultraviolet ray promotes destruction of constituents inside skin. Typically, the elevated level of active oxygen species is responsible for destruction of cells in skin and promotion of collagenase biosynthesis, resulting in degradation of collagen and elastin.

Collagen is an insoluble protein generated and secreted from dermis, playing an important role in maintaining skin elasticity. For maintaining skin elasticity, the generation and degradation of collagen must occur in normal manner. With skin aging, collagen is likely to abnormally generate and degrade. For collagen biosynthesis, Vitamin C and various extracts from oriental medicines (e.g., ginseng and chestnut shell) have been proposed and used as active ingredient in cosmetic compositions.

As discussed previously, materials promoting collagen biosynthesis are significantly applicable to and practically employed in cosmetic compositions for improving skin wrinkle.

As examples of cosmetic compositions for improving skin wrinkle, Korean Pat. Appln. No. 1996-000388 discloses a cosmetic composition for improving skin wrinkle comprising Vitamins A and E and aloe, Korean Pat. Appln. No. 2000-05223 suggests a cosmetic composition for improving skin wrinkle comprising antler, natural-occurring silt ingredients and Houttuynia Cordata and Korean Pat. Appln. No. 1999-031433 discloses a cosmetic cream composition for improving skin wrinkle comprising as active ingredient powderized extract from red grape.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

Having made intensive investigations on developing novel active ingredients for improving skin wrinkles, the present inventors have discovered that Mallotus japonicus extract exerts effective actions on elastase activity and collagen biosynthesis responsible for skin wrinkles and exhibits clinically excellent efficacy in improvement of skin wrinkles.

Accordingly, it is an object of this invention to provide a composition for inhibiting an activity of elastase.

It is another object of this invention to provide a composition for enhancing collagen biosynthesis.

It is still another object of this invention to provide a cosmetic composition for improving skin wrinkles.

Other objects and advantages of the present invention will become apparent from the detailed description to follow taken in conjugation with the appended claims.

### DETAILED DESCRIPTION OF THIS INVENTION

In one aspect of this invention, there is provided a composition for inhibiting an activity of elastase, which comprises a Mallotus japonicus extract as an active ingredient.

In another aspect of this invention, there is provided a composition for promoting collagen biosynthesis, which comprises a Mallotus japonicus extract as an active ingredient.

In still another aspect of this invention, there is provided a cosmetic composition for improving skin wrinkles comprising: (a) a Mallotus japonicus extract as an active ingredient; and (b) a cosmetically acceptable carrier.

In an effort to follow the above need in the art, the present inventors have strived to screen a wide variety of substances to develop an ingredient having excellent effect on improvement of skin wrinkles. In particular, the present inventors have focused on developing novel substances to inhibit elastase activity involved in formation of skin wrinkles as well as to promote collagen biosynthesis having pivotal role in improvement of skin wrinkles. As a result, the present inventors have discovered that Mallotus japonicus extract exhibits the above activities responsible for improvement of skin wrinkles.

Mallotus japonicus is a deciduous arborescent belonging to Euphobiaceae, mainly growing in the southern areas of Korea. Although its height sometimes reaches to 10 meter, it typically shows shrub appearance. Barks of Mallotus japonicus have been generally used as treating agent of gastric ulcer, gastritis and anhepatia.

The term used herein, “Mallotus japonicus extract” refers to that obtained from various organs and parts of Mallotus japonicus (e.g., leaf, flower, root, stem, branch, bark and fruit), preferably, root, stem, branch and bark, more preferably, root, branch and bark, and most preferably, bark.

In a preferred embodiment, Mallotus japonicus extract may be obtained using various extraction solvents, e.g. (a) water, (b) absolute or hydrous lower alcohol containing 1-4 carbon atoms (methanol, ethanol, propanol, butanol, etc.), (c) mixture of lower alcohol and water, (d) acetone, (e) ethyl acetate, (f) chloroform, (g) butyl acetate and (h) 1,3-butyleneglycol. The suitable amount of extraction solvent for obtaining Mallotus japonicus extract is 1-20 (preferably 1-10) fold higher than dry weight of Mallotus japonicus.

According to an exemplified process, the extract may be obtained as follows: Mallotus japonicus washed with distilled water and dried is cleaved into small pieces and 1-10-fold amount of extraction solvent is added. Under conditions to prevent the evaporation of active ingredients with cooling condenser, Mallotus japonicus immersed in solvent is heated at 40-100° C. for 3-20 hr for extraction. Alternatively, Mallotus japonicus immersed in solvent is heated at 4-40° C. for 1-15 days for extraction and the resultant is completely dried in a rotary vacuum evaporator. Where 1,3-butylene glycol is used as extraction solvent, the extraction is performed at low temperature as above and then the reduced dry weight of extract is adjusted to 1% (w/v).

Furthermore, it is apparent to one skilled in the art that other conventional solvents may be employed for yielding Mallotus japonicus extract showing substantially identical effects to that described above.

The extracts of Mallotus japonicus according to the present invention include those subject to additional purification by the well-known methods in the art as well as those obtained by extraction. For instance, it could be appreciated that active fractions obtained using a variety of additional purification methods such as an ultrafiltration with defined molecular weight cut-off value and various chromatography (designed for purification dependent upon size, charge, hydrophobicity and affinity) are included in the present extracts.

Mallotus japonicus extract according to the present invention can be powdered through additional processes such as lyophilization and spray drying.

According to the preferred embodiment of the present invention, the effective amount of Mallotus japonicus extract in cosmetic composition is 0.0001-20 wt %, more preferably 0.01-10 wt %, still more preferably 0.1-5 wt % and most preferably 1-3 wt % based on the total weight of the cosmetic composition. If the amount of Mallotus japonicus extract is lower than 0.0001 wt %, the effect of improving skin wrinkles may be negligible; in the case of exceeding 20 wt %, some adverse effects such as skin irritation and instability in formulation are very likely to occur.

Furthermore, the cosmetic compositions of the present invention may contain auxiliaries as well as carrier in addition to Mallotus japonicus extract. The non-limiting examples of auxiliaries include preservatives, antioxidants, stabilizers, solubilizers, Vitamins, colorants, odor improvers or mixtures of these ingredients.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, microcrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

The cosmetic compositions of this invention have novel use to improve skin wrinkles. The present compositions exhibit the inhibition effect on elastase activity and promotion effect on collagen biosynthesis at a molecular level, and as a result, excellent efficacy in improvement of skin wrinkles. Such effects and efficacies are demonstrated in Examples described hereunder. It would be appreciated that the improvement of skin wrinkles covers general uses of skin protection (e.g., prevention of skin wrinkles, removal of skin wrinkles and prevention of skin aging).

The following specific examples are intended to be illustrative of the invention and should not be construed as limiting the scope of the invention as defined by appended claims.

### EXAMPLE 1

Two hundreds g of Mallotus japonicus barks washed with distilled water and dried were added in 1.2 L of water, heated to 70-90° C. for 5 hours in extractor equipped with a cooling condenser, filtered through 300 mesh filter cloth and kept to stand at 5-10° C. for 7-10 days to ripen and filtered through Whattman No. 5 filter paper. The filtrate was dried in a rotary vacuum evaporator at 65° C., obtaining 5.9 g of dried powder of Mallotus japonicus.

### EXAMPLE 2

200 g of Mallotus japonicus barks washed with DW and dried were immersed in 1.2 L of water at 15-35° C. for 5 days, filtered through 300 mesh filter cloth and filtered again through Whattman No. 5 filter paper and then dried in a rotary vacuum evaporator to concentrate to 2-fold concentration. 0.6 L of 100% ethanol being added, it was kept to stand at 5-10° C. for 7-10 days to ripen and filtered through Whattman No. 5 filter paper. The filtrate was dried in a rotary vacuum evaporator at 65° C., yielding 5.8 g of dried powder of Mallotus japonicus extract.

### EXAMPLE 3

200 g of Mallotus japonicus barks washed with DW and dried was immersed in 1.2 L of water at 4-40° C. for 5 days, filtered through 300 mesh filter cloth, kept to stand at 5-10° C. for 7-10 days to ripen and filtered through Whattman No. 5 filter paper. The filtrate was dried in a rotary vacuum evaporator at 65° C., yielding 5.4 g of dried powder of Mallotus japonicus extract.

### EXAMPLES 4-21

The same processes as the above Example 3 were performed to produce Mallotus japonicus extract using extraction solvents described in Table 1. The results are represented in Table 1.

TABLE 1

| | | Dry weight of |
|---|---|---|
| Example | Extraction solvent | final extract (g) |
| Example 4 | 10% ethanol | 4.8 |
| Example 5 | 20% ethanol | 4.9 |
| Example 6 | 30% ethanol | 5.2 |
| Example 7 | 40% ethanol | 5.3 |
| Example 8 | 50% ethanol | 5.1 |
| Example 9 | 60% ethanol | 5.3 |
| Example 10 | 70% ethanol | 5.8 |
| Example 11 | 80% ethanol | 5.3 |
| Example 12 | 90% ethanol | 5.5 |
| Example 13 | 100% ethanol | 5.4 |
| Example 14 | Methanol | 5.3 |
| Example 15 | n-propanol | 4.5 |
| Example 16 | Isopropanol | 4.6 |
| Example 17 | 2-butanol | 4.2 |
| Example 18 | Acetone | 3.5 |
| Example 19 | Chloroform | 3.2 |
| Example 20 | Ethylacetate | 3.3 |
| Example 21 | Butylacetate | 3.6 |

### EXAMPLE 22

200 g of Mallotus japonicus barks washed with DW and dried was immersed in 1.2 L of 1,4-butyleneglycol for 48 hours, filtered through 300 mesh filter cloth, kept to stand at 5-10° C. for 7-10 days to ripen and filtered through Whattman No. 5 filter paper. The extract was then calculated for reduced dry weight to adjust the final concentration to 1% (w/v) due to.

### EXAMPLE 23

200 g of Mallotus japonicus barks washed with DW and dried was added in 1.2 L of 10% ethanol, heated for 5 hours in extractor equipped with a cooling condenser, filtered through 300 mesh filter cloth and kept to stand at 5-10° C. for 7-10 days to ripen and filtered through Whattman No. 5 filter paper. The extract was then dried in a rotary vacuum evaporator at 65° C., giving 6.2 g of dried powder of Mallotus japonicus.

### EXAMPLES 24-32

The same processes as the above Example 23 were carried out to obtain Mallotus japonicus extracts using extraction solvents indicated in Table 2. The results are found in Table 2.

TABLE 2

| | | Dry weight of |
|---|---|---|
| Example | Extraction solvent | final extract (g) |
| Example 24 | 20% ethanol | 6.3 |
| Example 25 | 30% ethanol | 6.5 |
| Example 26 | 40% ethanol | 6.4 |
| Example 27 | 50% ethanol | 6.3 |
| Example 28 | 60% ethanol | 6.1 |
| Example 29 | 70% ethanol | 6.8 |
| Example 30 | 80% ethanol | 6.2 |
| Example 31 | 90% ethanol | 6.2 |
| Example 32 | 100% ethanol | 6.0 |

### EXPERIMENTAL EXAMPLE 1

### Inhibitory Effect on Elastase Activity

The inhibitory effect of Mallotus japonicus extracts prepared in Examples 1-32 on elastase activity was tested as follows:

Each of Mallotus japonicus extracts was used in the amount of 100 μg/ml. 20 μl of substrate solution (to 8.8 mM elastase substrate Succ-Ala-Ala-Ala-p-nitroanilide standard solution, Sigma) were added to 60 μl of buffer (0.267 M Tris adjusted to pH 8.0 with 0.267 M HCl), and then 100 μl of Mallotus japonicus extract were added and 20 μl of enzyme solution (10 μg/ml of porcine pancreas elastase standard, Sigma) were added to react at 25° C. for 15 min. The absorbance was measured at 410 nm. The control group contains distilled water instead of Mallotus japonicus extracts. Inhibitory rate against elastase activity was calculated according to the following equation: Inhibitory rate against elastase activity (%)={(absorbance of test group−absorbance of control group)/absorbance of control group}×100

TABLE 3
Inhibitory Effect of Mallotus japonicus Extract on
Elastase Activity (test conc. 100 μg/ml)

| | Inhibitory rate against |
|---|---|
| Extract Tested | elastase activity (%) |
| Example 1 | 65.3 |
| Example 2 | 63.5 |
| Example 3 | 64.8 |
| Example 4 | 66.3 |
| Example 5 | 65.8 |
| Example 6 | 67.5 |
| Example 7 | 68.5 |
| Example 8 | 69.6 |
| Example 9 | 71.2 |
| Example 10 | 73.2 |
| Example 11 | 73.3 |
| Example 12 | 74.2 |
| Example 13 | 73.5 |
| Example 14 | 75.6 |
| Example 15 | 76.8 |
| Example 16 | 77.5 |
| Example 17 | 73.2 |
| Example 18 | 72.8 |
| Example 19 | 71.5 |
| Example 20 | 70.5 |
| Example 21 | 68.9 |
| Example 22 | 65.8 |
| Example 23 | 66.5 |
| Example 24 | 63.5 |
| Example 25 | 62.5 |
| Example 26 | 63.6 |
| Example 27 | 68.9 |
| Example 28 | 65.6 |
| Example 29 | 62.3 |
| Example 30 | 61.2 |
| Example 31 | 65.3 |
| Example 32 | 62.3 |

As shown in Table 3, it could be realized that Mallotus japonicus extract exhibits considerable inhibitory effect on elastase activity, although it shows slightly different inhibition pattern depending on extraction process.

### EXPERIMENTAL EXAMPLE 2

### Analysis of Effect on Collagen Biosynthesis

Human normal fibroblasts available from the Korean Cell Line Research Foundation were inoculated (1×10⁶ cells/well) on 96-well microplate containing DMEM and incubated for 24 hours at 37° C. After incubation, the medium in microplate was replaced with fresh serum-free DMEM containing 100 ppm Mallotus japonicus extract and then additional incubation for 24 hours was carried out. Control contained no Mallotus japonicus extract. Following incubation, procollagen type IC-peptide (PICP) obtained from the supernatant of each well was measured using a kit purchased from Takara Co. (Japan) and the measured values were converted to ng/ml, which indicate the amount of collagen newly generated. The results are summarized in Table 4.

TABLE 4
Effect of Mallotus japonicus Extract on Collagen
Biosynthesis

| | Effect on collagen |
|---|---|
| Extract Tested | biosynthesis (%) |
| Example 1 | 72.3 |
| Example 2 | 73.5 |
| Example 3 | 74.2 |
| Example 4 | 73.2 |
| Example 5 | 71.2 |
| Example 6 | 70.5 |
| Example 7 | 70.6 |
| Example 8 | 68.9 |
| Example 9 | 75.3 |
| Example 10 | 72.5 |
| Example 11 | 73.5 |
| Example 12 | 71.2 |
| Example 13 | 73.2 |
| Example 14 | 71.0 |
| Example 15 | 72.2 |
| Example 16 | 73.3 |
| Example 17 | 71.2 |
| Example 18 | 71.5 |
| Example 19 | 72.5 |
| Example 20 | 71.6 |
| Example 21 | 70.2 |
| Example 22 | 75.3 |
| Example 23 | 70.6 |
| Example 24 | 70.5 |
| Example 25 | 70.3 |
| Example 26 | 70.5 |
| Example 27 | 75.2 |
| Example 28 | 74.3 |
| Example 29 | 73.5 |
| Example 30 | 76.8 |
| Example 31 | 72.5 |
| Example 32 | 71.5 |

As shown in Table 4, it could be understood that Mallotus japonicus extract exhibits significant enhancing effect on collagen biosynthesis, although it shows slightly different enhancing pattern depending on extraction process.

The following formulations are illustrative and the cosmetic composition of the invention are not restricted by them. Mallotus japonicus extract used is that obtained in Example 10.

### Formulation I: Skin Lotion (Skin Softener)

One example of skin lotions containing Mallotus japonicus extract is formulated as Table 5:

TABLE 5

| Ingredients | Amount (wt %) |
|---|---|
| Mallotus japonicus extract | 2.0 |
| Glycerine | 5.0 |
| 1,3-butylglycol | 3.0 |
| PEG 150 | 1.0 |
| Alantoine | 0.1 |
| DL-pantenol | 0.3 |
| EDTA-2Na | 0.02 |
| Benzophenon-9 | 0.04 |
| Sodium hyaluronate | 5.0 |
| Ethanol | 10.0 |
| Octyldodeces-16 | 0.2 |
| Polysorbate 20 | 0.2 |
| Antiseptic, fragrant, colorant | Minute amount |
| DW | Residual amount |
| Total | 100 |

### Formulation 2: Astringent Lotion

One example of astringent lotions containing Mallotus japonicus extract is formulated as Table 6:

TABLE 6

| Ingredients | Amount (wt %) |
|---|---|
| Mallotus japonicus extract | 2.0 |
| Glycerine | 2.0 |
| 1,3-butylglycol | 2.0 |
| Alantoine | 0.2 |
| DL-pantenol | 0.2 |
| EDTA-2Na | 0.02 |
| Benzophenon-9 | 0.04 |
| Sodium hyaluronate | 3.0 |
| Ethanol | 15.0 |
| Polysorbate 20 | 0.3 |
| Witchhazel extract | 2.0 |
| Citric acid | Minute amount |
| Antiseptic, fragrant, colorant | Minute amount |
| DW | Residual amount |
| Total | 100 |

### Formulation 3: Nutrient Emulsion (Milk Lotion)

One example of nutrient lotions containing Mallotus japonicus extract is formulated as below Table 7:

TABLE 7

| Ingredients | Amount (wt %) |
|---|---|
| Mallotus japonicus extract | 2.0 |
| Glyceryl stearate SE | 1.5 |
| Stearyl alcohol | 1.5 |
| Lanoline | 1.5 |
| Polysorbate 60 | 1.3 |
| Sorbitan stearate | 0.5 |
| Hydrogenated vegetable oil | 1.0 |
| Mineral oil | 5.0 |
| Squalane | 3.0 |
| Trioctanoine | 2.0 |
| Dimethicon | 0.8 |
| Tocopherol acetate | 0.5 |
| Carboxyvinyl polymer | 0.12 |
| Glycerine | 5.0 |
| 1,3-butylglycol | 3.0 |
| Sodium hyaluronate | 5.0 |
| Tri-ethanolamine | 0.12 |
| Antiseptic, fragrant, colorant | Minute amount |
| DW | Residual amount |
| Total | 100 |

### Formulation 4: Nutrient Cream

One example of nutrient creams containing Mallotus japonicus extract is formulated as Table 8:

TABLE 8

| Ingredients | Amount (wt %) |
|---|---|
| Mallotus japonicus extract | 2.0 |
| Lypophilic glycerol monostearate | 2.0 |
| Cetearyl alcohol | 2.2 |
| Stearic acid | 1.5 |
| Wax | 1.0 |
| Polysorbate 60 | 1.5 |
| Sorbitan stearate | 0.6 |
| Hydrogenated vegetable oil | 1.0 |
| Squalane | 3.0 |
| Mineral oil | 5.0 |
| Trioctanoine | 5.0 |
| Dimethicon | 1.0 |
| Sodium magnesium silicate | 0.1 |
| Glycerine | 5.0 |
| Betaine | 3.0 |
| Tri-ethanolamine | 1.0 |
| Sodium hyaluronate | 4.0 |
| Antiseptic, fragrant, colorant | Minute amount |
| DW | Residual amount |
| Total | 100 |

### Formulation V: Message Cream

One example of message creams containing Mallotus japonicus extract is formulated as Table 9:

TABLE 9

| Ingredients | Amount (wt %) |
|---|---|
| Mallotus japonicus extract | 2.0 |
| Lypophilic glycerol monostearate | 1.5 |
| Stearyl alcohol | 1.5 |
| Stearic acid | 1.0 |
| Polysorbate 60 | 1.5 |
| Sorbitan stearate | 0.6 |
| Isostearyl isostearate | 5.0 |
| Squalane | 5.0 |
| Mineral oil | 35.0 |
| Dimethicon | 0.5 |
| Hydroxyethyl cellulose | 0.12 |
| Glycerine | 6.0 |
| Tri-ethanolamine | 0.7 |
| Antiseptic, fragrant, colorant | Minute amount |
| DW | Residual amount |
| Total | 100 |

### Formulation 6: Essence

One example of essence containing Mallotus japonicus extract is formulated as Table 10:

TABLE 10

| Ingredients | Amount (wt %) |
|---|---|
| Bergenia emeiensis extract | 2.0 |
| Glycerine | 10.0 |
| Betaine | 5.0 |
| PEG 1500 | 2.0 |
| Alantoine | 0.1 |
| DL-pantenol | 0.3 |
| EDTA-2Na | 0.02 |
| Benzophenon-9 | 0.04 |
| Hydroxyethyl cellulose | 0.1 |
| Sodium hyaluronate | 8.0 |
| Carboxyvinyl polymer | 0.2 |
| Triethanolamine | 0.18 |
| Octyldodecanol | 0.3 |
| Octyldodeces-16 | 0.4 |
| Ethanol | 6.0 |
| Antiseptic, fragrant, colorant | Minute amount |
| DW | Residual amount |
| Total | 100 |

### Formulation 7: Facial Pack

One example of facial packs containing Mallotus japonicus extract is formulated as Table 11:

TABLE 11

| Ingredients | Amount (wt %) |
|---|---|
| Bergenia emeiensis extract | 2.0 |
| Polyvinyl alcohol | 15.0 |
| Cellulose gum | 0.15 |
| Glycerine | 3.0 |
| PEG 1500 | 2.0 |
| Cyclodextrin | 0.15 |
| DL-pantenol | 0.4 |
| Alantoine | 0.1 |
| Monoammonium glycyrrhizinate | 0.3 |
| Nicotineamide | 0.5 |
| Ethanol | 6.0 |
| PEG 40 hydrogenated castor oil | 0.3 |
| Antiseptic, fragrant, colorant | Minute amount |
| DW | Residual amount |
| Total | 100 |

### EXPERIMENTAL EXAMPLE 3

### Efficacy of Cosmetic Compositions of the Invention on Improving Skin Wrinkles

The efficacy of the cosmetic compositions on improving skin wrinkles was evaluated through practical applications. The nutrient cream containing 2% Mallotus japonicus extract described in Formulation 4 was employed and Mallotus japonicus extract was replaced by the same amount of DW for control in this trial. At first, 20 women aged 30-40 randomized 2 groups were administered with the nutrient cream of Formulation 4 or its control cream. The application in eye rims lasted for 2 months with diurnal twice applications in every morning and night. Improving efficacy of skin wrinkles was evaluated by observation compared to control groups. The results are summarized in the following Table 12:

TABLE 12

| | | Moderately | | Efficacy |
|---|---|---|---|---|
| Formulation | Effective | effective | Ineffective | (%) |
| Formulation 4 | 10 | 4 | 6 | 70.0 |
| Control | 4 | 5 | 12 | 45.0 |

As shown in Table 12, Formulation 4 according to the present invention shows significantly enhanced improving effect of skin wrinkles compared to its control formulation. Furthermore, there was no skin trouble in any testee administered with the present formulation.

Having described preferred embodiments of the present invention, it is to be understood that variants and modifications thereof falling within the spirit of the invention may become apparent to those skilled in this art, and the scope of this invention is to be determined by appended claims and their equivalents.

We claim:

1. A method of improving skin wrinkle in a skin comprising administering to said skin in an amount of 0.01-10 wt % of Mallotous japonicus extract based on the total weight

2. The method of claim 1 wherein the Mallotous japonicus extract is present in an amount of 0.1-5 wt % based on the total weight.

3. The method of claim 1 wherein the Mallotous japonicus extract is prepared in the form of one selected from a solution, a suspension, an emulsion, a paste, an ointment, a gel, a powder, a soap, a surfactant-containing cleanser, an oil, a powder foundation, an emulsion foundation, a wax foundation and a spray.

4. A cosmetic treatment method for improving skin wrinkles comprising applying to said skin wrinkles a cosmetic composition comprising an 0.01-10 wt % of an extract of Mallotous japonicus based on the total weight of said composition.

5. The method of claim 4 wherein said skin wrinkles are face, neck or hand wrinkles.

6. The method of claim 5 wherein said face wrinkles are wrinkles around the eyes.

7. The method of claim 6 wherein said composition contains 0.1-1.5 wt % of said extract based on the total weight of said composition.

8. The method of claim 6 wherein said composition is in the form of a solution, a suspension, an emulsion, a paste, an ointment, a gel, a powder, a soap, a surfactant-containing cleanser, an oil, a powder foundation, an emulsion foundation, a wax foundation or a spray.
